(19) United States
(12) Patent Application Publication
(10) Pub. No.: US 2026/0325056 A1
(43) Pub. Date: Oct. 22, 2026

(54) GAS SUPPLY CONTROL METHOD AND GAS SUPPLY CONTROL SYSTEM

(71) Applicants: Haruhiro INOUE, Tokyo (JP); OLYMPUS MEDICAL SYSTEMS CORP., Tokyo (JP)

(72) Inventors: Haruhiro INOUE, Tokyo (JP); Masahiko OKAMURA, Tokyo (JP); Kayuri KIMURA, Tokyo (JP)

(73) Assignees: Haruhiro INOUE, Tokyo (JP); OLYMPUS MEDICAL SYSTEMS CORP., Tokyo (JP)

(21) Appl. No.: 19/693,979

(22) Filed: Jun. 1, 2026

Related U.S. Application Data
(63) Continuation of application No. PCT/JP2023/043285, filed on Dec. 4, 2023.

(51) Int. Cl. A61B 5/00

(52) U.S. Cl. CPC A61B 5/4211; A61B 5/742

(57) ABSTRACT

A gas supply control method implemented by processing circuitry operatively coupled to a gas feeding assembly includes causing the gas feeding assembly to supply gas to a lumen of a subject at a first flow rate. The gas supply control method further includes obtaining a pressure of the gas within the lumen, causing the gas feeding assembly to supply the gas to the lumen at a second flow rate less than the first flow rate when the obtained pressure is equal to or greater than a first pressure, and causing the gas feeding assembly to cease supplying the gas to the lumen when the obtained pressure is equal to or greater than a second pressure greater than the first pressure.

### CROSS REFERENCE TO RELATED APPLICATION

This application is a continuation of International Application No. PCT/JP2023/043285, filed on Dec. 4, 2023, the entire contents of which are incorporated herein by reference.

### FIELD

Embodiments of the present disclosure relate to a gas supply control method for supplying gas to a lumen of a subject, and a gas supply control system for supplying gas to the lumen of the subject.

### BACKGROUND

Gastroesophageal reflux disease (hereinafter referred to as “GERD”) is a functional disorder where, for example, the function of the gastric cardia deteriorates, causing gastric fluid to reflux to the esophagus. As methods for diagnosing whether a subject has GERD (hereinafter referred to as “GERD diagnostic methods”), techniques such as 24-hour esophageal pH monitoring and 24-hour esophageal impedance pH monitoring are used.

To mitigate the burden on the examinee in GERD diagnostic methods, a method using an endoscope to observe the relaxation state of the lower esophageal sphincter (LES) at the gastric cardia is increasingly being employed.

International Publication No. 2021/166127 discloses an endoscope system that diagnoses GERD based on pressure change patterns by measuring the pressure within a lumen of a subject while supplying gas to the lumen.

Unlike GERD, esophageal achalasia, where an abnormality occurring in the function of the lower esophageal sphincter causes difficulty in passing food from the esophagus to the stomach, is also one of the esophageal functional disorders.

### SUMMARY

According to aspects of the present disclosure, a gas supply control method is provided that is implemented by processing circuitry operatively coupled to a gas feeding assembly. The gas supply control method includes causing the gas feeding assembly to supply gas to a lumen of a subject at a first flow rate. The gas supply control method further includes obtaining a pressure of the gas within the lumen. The gas supply control method further includes causing the gas feeding assembly to supply the gas to the lumen at a second flow rate less than the first flow rate when the obtained pressure is equal to or greater than a first pressure. The gas supply control method further includes causing the gas feeding assembly to cease supplying the gas to the lumen when the obtained pressure is equal to or greater than a second pressure greater than the first pressure.

According to aspects of the present disclosure, further provided is a gas supply control method implemented by processing circuitry operatively coupled to a gas feeding assembly. The gas supply control method includes causing the gas feeding assembly to supply gas to a lumen of a subject at a first flow rate. The gas supply control method further includes obtaining a pressure of the gas within the lumen. The gas supply control method further includes causing the gas feeding assembly to supply the gas to the lumen at a second flow rate less than the first flow rate when at least one of a first condition or a second condition is satisfied. The first condition is a condition in which the obtained pressure is equal to or greater than a first pressure. The second condition is a condition in which a rising rate of the obtained pressure exceeds a predetermined rising rate.

According to aspects of the present disclosure, further provided is a gas supply control system that includes a first tube, a pressure sensor, a second tube, a valve, and processing circuitry. The first tube is configured to communicate with a lumen of a subject. The pressure sensor is disposed at the first tube and configured to measure a pressure of gas within the lumen. The second tube is configured to communicate with the lumen and configured to supply the gas from a gas supply source to the lumen. The valve is configured to adjust a flow rate of the gas supplied through the second tube. The processing circuitry is configured to control the valve to supply the gas to the lumen at a first flow rate. The processing circuitry is further configured to obtain the pressure measured by the pressure sensor. The processing circuitry is further configured to control the valve to supply the gas to the lumen at a second flow rate less than the first flow rate when at least one of a first condition or a second condition is satisfied. The first condition is a condition in which the obtained pressure is equal to or greater than a first pressure. The second condition is a condition in which a rising rate of the obtained pressure exceeds a predetermined rising rate.

### BRIEF DESCRIPTION OF DRAWINGS

FIG. 1 is a general configuration diagram of a gas supply control system including a gas supply control apparatus according to an embodiment;

FIG. 2 is a configuration diagram of the gas supply control system including the gas supply control apparatus according to the embodiment;

FIG. 3 is a flowchart of a pressure control method of the embodiment;

FIG. 4 is a graph showing pressure changes in a lumen of a patient with GERD according to the embodiment;

FIG. 5 is a graph showing pressure changes in a lumen of a healthy subject according to conventional apparatuses;

FIG. 6 is a graph showing pressure changes in a lumen of a healthy subject according to the embodiment;

FIG. 7 is a graph showing pressure changes in a lumen of a patient with achalasia according to the embodiment;

FIG. 8 is a flowchart of a pressure control method of a gas supply control system according to modification 1 of the embodiment;

FIG. 9 is a graph showing pressure changes in a lumen of a healthy subject, according to modification 1 of the embodiment;

FIG. 10 is a graph showing pressure changes according to modification 2 of the embodiment; and

FIG. 11 is a graph showing pressure changes in a lumen of a healthy subject, according to modification 3 of the embodiment.

### DESCRIPTION OF EMBODIMENTS

Hereinafter, embodiments will be described with reference to the drawings. It should be noted that the drawings based on the embodiments are schematic. The relationship between the thickness and width of each part and the ratio of the thickness of each part in the drawings differ from the actual ones. The drawings also contain parts in which dimensional relationships and ratios are different from each other. Illustration of some components and their reference numerals are omitted.

### Embodiments

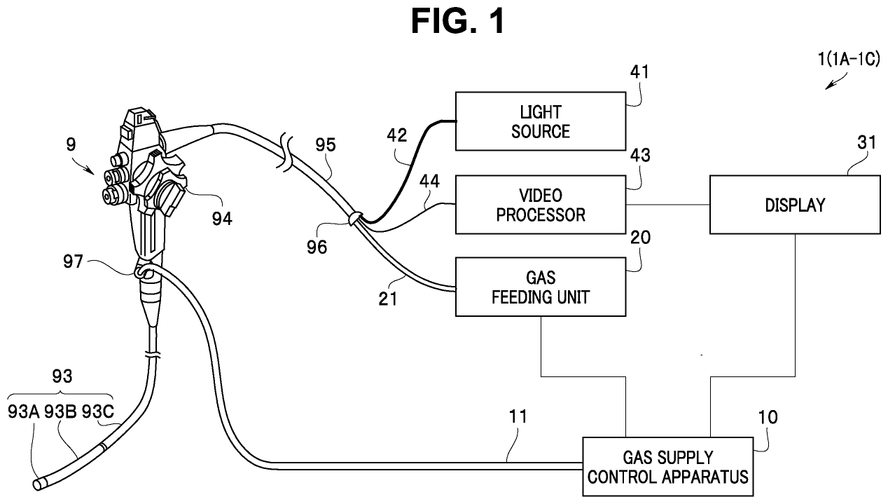

A gas supply control system 1 of an embodiment shown in FIG. 1 includes an endoscope 9, a gas supply control apparatus 10, a gas feeding unit (hereinafter also referred to as a gas feeding assembly) 20, a display 31, a video processor 43, and a light source 41. The endoscope 9, the gas supply control apparatus 10, the gas feeding unit 20, the display 31, the video processor 43, and the light source 41 are hardware.

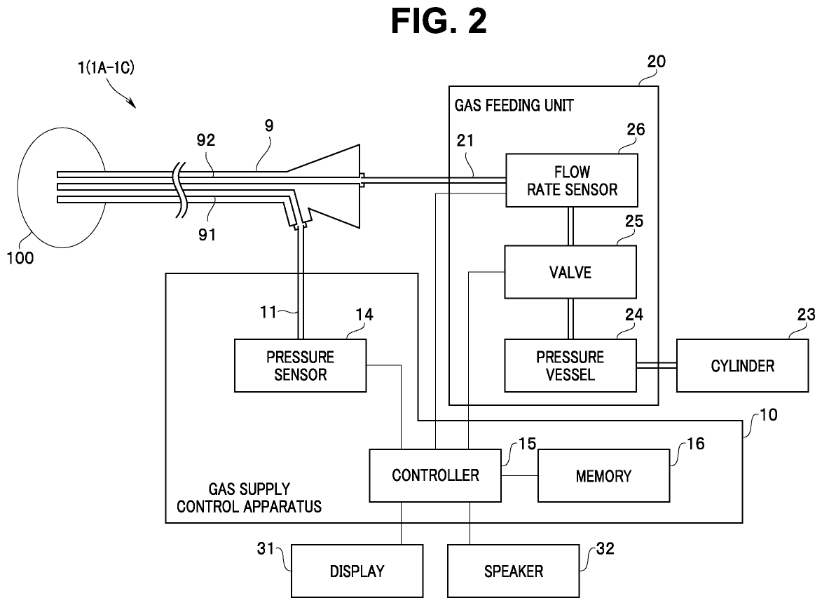

The endoscope 9 includes an elongated insertion portion 93 to be inserted into a subject, an operation portion 94 provided at a proximal end of the insertion portion 93, a universal cord 95 extending from the operation portion 94, and a connector 96. The operation portion 94 includes a bending operation knob and a plurality of buttons for operating endoscope functions. The insertion portion 93 of the endoscope 9 includes, in order from a distal end side, a distal end portion 93A, a bendable bending portion 93B provided at a proximal end of the distal end portion 93A, and an elongated flexible pipe 93C provided at a proximal end of the bending portion 93B. Although not shown, the distal end portion 93A is provided with an image pickup unit, an illumination optical system, and an opening of a forceps channel 91 (FIG. 2). The forceps channel 91 is a tube passing through the insertion portion 93 from an insertion port 97 of the operation portion 94 to the opening of the distal end portion 93A.

The connector 96 connects to an optical fiber bundle 42 of the light source 41, a cable 44 of the video processor 43, and a second tube (gas feeding tube) 21 of the gas feeding unit 20. Illumination light generated by the light source 41 is guided via the optical fiber bundle 42 to the illumination optical system of the distal end portion 93A. The gas feeding unit 20 ejects, in response to a button operation of the operation portion 94, a fluid (gas or liquid) from the opening of the distal end portion 93A via the gas/liquid feeding pipe 92 (FIG. 2) passing through the universal cord 95, the operation portion 94, and the insertion portion 93. That is, the gas feeding unit 20 is a gas/liquid feeding unit capable of supplying not only gas but also liquid, but in the description of the present disclosure, the fluid used is only gas. Gas/liquid feeding is switched by a button operation of the operation portion 94. The second tube 21 may be connected to the operation portion 94.

The video processor 43 processes an image pickup signal from the image pickup unit of the distal end portion 93A and displays an endoscopic image on the display 31.

The gas supply control system 1 of the present embodiment is configured by adding the gas supply control apparatus 10 to an endoscope system mentioned above.

As shown in FIG. 2, the gas supply control apparatus 10 includes a first tube 11, a pressure sensor 14, a controller 15, and a memory 16. The first tube 11, the pressure sensor 14, the controller 15, and the memory 16 are hardware.

The first tube 11 is connected to the insertion port 97 of the forceps channel 91 of the endoscope 9. When the distal end portion 93A of the insertion portion 93 of the endoscope 9 is inserted into the stomach 100, which is a lumen of the subject, the first tube 11 communicates with the interior of the stomach 100 via the forceps channel 91. The pressure sensor 14 is disposed on the first tube 11. The pressure sensor 14 measures a pressure of gas within the first tube 11. Since the first tube 11 communicates with the stomach 100, the pressure sensor 14 measures a pressure of the gas within the stomach 100.

The controller 15 may be implemented by processing circuitry including one or more processors and non-transitory computer-readable storage media. For example, the controller 15, including a central processing unit (CPU), performs control of the entirety of the gas supply control system 1. In this case, for instance, the controller 15 may be configured to take overall control of the gas supply control system 1 by one or more processors (e.g., the CPU) executing programs stored in one or more non-transitory computer-readable storage media. The memory 16 stores setting conditions and the like for the gas supply control system 1.

The gas feeding unit 20 includes the second tube 21, a flow rate sensor 26, a pressure vessel 24, and a valve 25. The second tube 21 is connected to the gas/liquid feeding pipe 92 of the endoscope 9. When the distal end portion 93A of the insertion portion 93 of the endoscope 9 is inserted into the stomach 100, which is a lumen of the subject, the second tube 21 communicates with the interior of the stomach 100 via the gas/liquid feeding pipe 92.

Gas, such as carbon dioxide gas, supplied from a cylinder 23, which is a gas supply source, is decreased in pressure by a regulator (not shown) and fed to the second tube 21 via the pressure vessel 24.

The gas supply control system 1 diagnoses GERD and achalasia by measuring a pressure (internal pressure) of gas within the stomach 100 using the pressure sensor 14, while feeding gas from the gas feeding unit 20 to the stomach 100 of the subject via the endoscope 9. Pressure data is displayed on the display 31 in a time series. The display 31 displays the pressure data together with an endoscopic image, but may be a display dedicated to pressure data. A speaker 32 notifies a user audibly, for example, of an abnormality occurrence.

As will be mentioned later, the controller 15 of the gas supply control system 1 of the embodiment starts supplying gas at a first flow rate F1, and when the internal pressure of the stomach 100 reaches a first pressure P1, the controller 15 supplies gas to the stomach 100 at a second flow rate F2 below the first flow rate F1. The first pressure P1 is a GERD diagnostic pressure serving as a criterion for GERD diagnosis determination. The first flow rate F1 is set, for example, greater than the flow rate of conventional gas supply control systems, and the second flow rate F2 is set, for example, less than the flow rate of conventional gas supply control systems. The first flow rate F1, the second flow rate F2, and the first pressure P1 are stored in the memory 16, for example.

For this reason, the gas supply control system 1 can perform a diagnosis in a shorter time period and more accurately than conventional gas supply control systems.

The above-mentioned configuration of the gas supply control system 1 is merely one example, and the present disclosure is not limited to the above-mentioned configuration.

For example, the valve 25 may be disposed at a position closer to the endoscope 9 than the pressure vessel 24 of the second tube 21. The gas supply control system may include a microphone for collecting sounds from the person (i.e., the examinee) under examination during the examination. Instead of detecting a pressure reduction caused by belching, the microphone detects a belching sound produced from the oral cavity of the examinee and the controller 15 performs control to stop gas feeding. The gas supply control system may include a video recorder and a video printer for recording images displayed on the display 31.

### Gas Supply Control Method

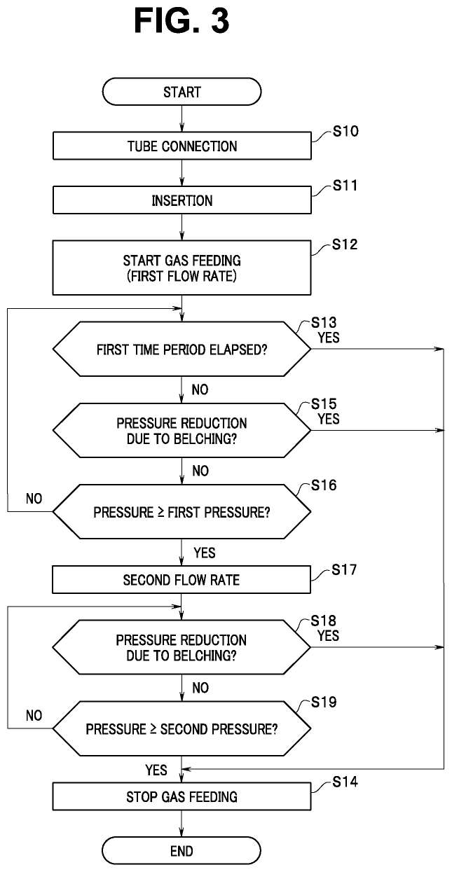

A gas supply control method by the gas supply control system 1 will be described along the flowchart of FIG. 3.

### <Step S10> Tube Connection

The first tube 11 is connected to the insertion port 97 of the forceps channel 91 of the endoscope 9. The second tube 21 is connected to the gas/liquid feeding pipe 92 of the endoscope 9.

### <Step S11> Insertion

The distal end portion 93A of the insertion portion 93 of the endoscope 9 is inserted into the interior of the stomach 100 of the subject. It should be noted that observation of the cardia may be performed using the image pickup unit disposed at the distal end portion 93A.

### <Step S12> Start Gas Feeding (First Flow Rate)

The controller 15 controls the valve 25 of the gas feeding unit 20 to feed gas into the stomach 100 at a first flow rate F1. The first flow rate F1 is, for example, 1.5 ml/s to 2.5 ml/s. The valve 25 is, for example, a type of solenoid valve, and includes a regulating valve using an electromagnetic coil as a drive section. When a current is caused to flow through the electromagnetic coil, a magnetic force is generated, attracting the plunger to open or close the valve. The magnitude of the current caused to flow through the electromagnetic coil controls an opening degree of the valve, adjusting a flow rate of gas to a predetermined value.

### <Step S13> First Time Period Elapsed?

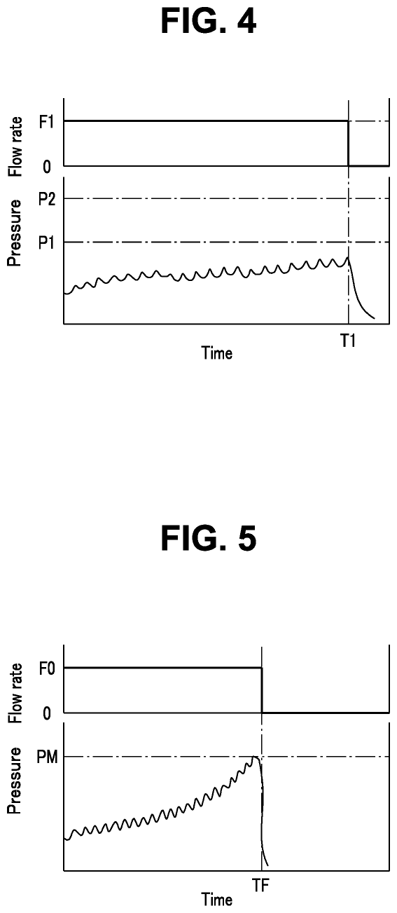

FIG. 4 shows changes in gastric internal pressure with respect to a time period during which gas is fed in a patient with GERD having deteriorated function of the gastric cardia. It should be noted that gastric internal pressure reflects respiratory variation, and rises during inspiration relative to expiration. For this reason, the measured pressure repeatedly increases and decreases according to the respiratory cycle.

In a patient with GERD, gas leaks from the cardia even when gas is fed into the stomach 100. For this reason, even after a predetermined first time period T1 has elapsed since gas feeding starts, the pressure P does not reach, for example, the first pressure P1, which is the GERD diagnostic pressure. The first time period T1 and the first pressure P1 are set as appropriate. For example, the first time period T1 is 5 minutes, and the first pressure P1 is greater than 16 mmHg and less than 19 mmHg.

When the time period from the start of gas feeding, or in other words, a time period during which gas is fed at the first flow rate F1 reaches the first time period T1 (S13: YES), the process proceeds to step S14.

It should be noted that the controller 15 may calculate the rising rate ΔP/s of the pressure P. If the rising rate ΔP/s is lower than a predetermined first rising rate ΔP1/s, to expedite the examination, the process may proceed to step S14 and end the examination, since GERD can be determined even before the first time period T1 elapses.

The first rising rate ΔP 1/s is set, for example, to 50% to 75% of the rising rate ΔP/s of a healthy subject. The first rising rate ΔP 1/s is, for example, 0.1 mmHg/s. To inhibit erroneous determination, it may be preferable to use, for calculating the rising rate ΔP/s, an average rising rate during an examination time period that is at least 20% greater than the first time period T1.

If the rising rate ΔP/s is lower than the predetermined first rising rate ΔP1/s, the controller 15 may decrease the gas feeding rate to a flow rate of 1.0 ml/s to 2.0 ml/s, provided that the flow rate is less than the first flow rate F1.

### <Step S14> Stop Gas Feeding

The gas feeding from the gas feeding unit 20 is stopped under control of the controller 15, and the process ends.

### <Step S15> Pressure Reduction Due To Belching?

In a patient with mild GERD, gas in the stomach 100, even at low internal pressure, may be emitted from the mouth via belching, causing the internal pressure to reduce sharply. If the controller 15 senses that the pressure has fallen sharply (S15: YES), the process proceeds to step S14. A sharp fall refers to, for example, a fall rate less than −5 mmHg/s (i.e., an absolute value of the fall rate exceeding 5 mmHg/s). It should be noted that, to inhibit a sharp decrease in flow rate when transitioning from step S13 or step S15 to S14 (stop gas feeding), the gas feeding may be reduced to a predetermined flow rate (for example, the second flow rate F2) and then stopped (S14).

### <Step S16> Pressure≥First Pressure?

The gas feeding at the first flow rate F1 continues until the pressure reaches the first pressure P1.

### <Step S17> Second Flow Rate

Here, FIG. 5 is a graph showing internal pressure changes in a healthy subject in conventional gas supply control systems. When gas is supplied to the stomach 100 at a flow rate F0, the internal pressure gradually increases, and upon reaching a standard pressure PM, the gas within the stomach 100 is emitted from the mouth via belching, causing the internal pressure to reduce sharply. The standard pressure PM of a healthy subject is, for example, greater than 18 mmHg and less than 25 mmHg.

However, in conventional gas supply control systems, if the flow rate F0 is small, a time period TF required for the internal pressure to reach the standard pressure PM becomes longer, prolonging the examination time period. On the other hand, if the flow rate F0 is large, an error in the time period TF for the internal pressure to reach the standard pressure PM becomes large, which may reduce the diagnostic accuracy.

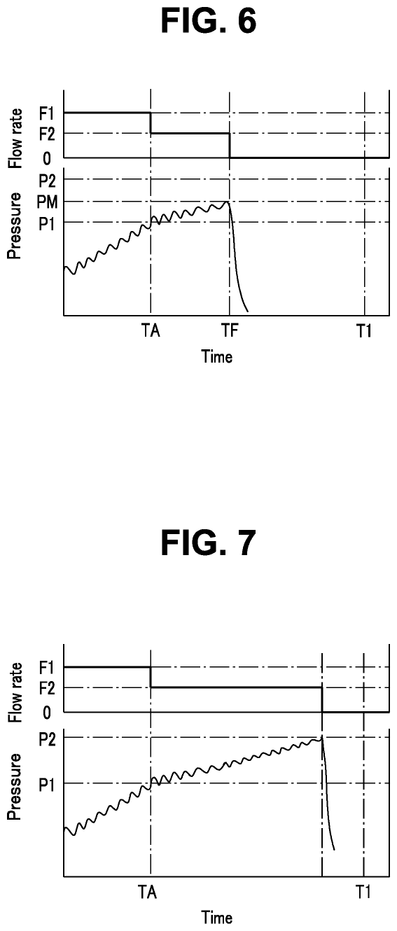

In contrast, as shown in FIG. 6, in the gas supply control system 1, when the pressure reaches the first pressure P1 (time TA), the flow rate of gas is decreased from the first flow rate F1 to the second flow rate F2. The second flow rate F2 may preferably be less than 75% of the first flow rate F1, and may particularly preferably be less than 50%. The second flow rate F2 is, for example, 0.5 ml/s to 1.5 ml/s.

That is, the first flow rate F1 is greater than the flow rate F0 in conventional gas supply control systems, and the second flow rate F2 is less than the flow rate F0.

It should be noted that, for some examinees, the rising rate ΔP/s of the pressure P may increase sharply during gas feeding. In such a case, even if the flow rate is dropped after the pressure reaches the first pressure P1, the error in the time period TF for the internal pressure to reach the standard pressure PM may become large.

The controller 15 of the gas supply control system 1 may calculate the rising rate ΔP/s of pressure, and when the rising rate ΔP/s exceeds a predetermined second rising rate ΔP2/s, the controller 15 may supply gas at the second flow rate F2 even if the pressure is equal to or less than the first pressure P1. In other words, the controller 15 may be configured to cause the gas feeding unit 20 to supply gas at the second flow rate F2 when at least one of a first condition or a second condition is satisfied. The first condition may be a condition in which the pressure is equal to or greater than the first pressure P1. The second condition may be a condition in which the rising rate ΔP/s exceeds the predetermined second rising rate ΔP2/s.

### <Step S18> Pressure Reduction Due To Belching?

In a healthy subject, when the internal pressure of the stomach 100 becomes higher than the standard pressure PM, the gas within the stomach 100 is emitted from the mouth via belching, causing the pressure to reduce sharply.

If the controller 15 senses that the pressure has fallen sharply (S18: YES), the process proceeds to step S14.

### <Step S19> Pressure≥Second Pressure?

The gas feeding is continued until the pressure reaches a second pressure P2 greater than the first pressure P1.

As shown in FIG. 7, in an examinee with achalasia having an abnormality occurring in the function of the lower esophageal sphincter, even when the pressure reaches the second pressure P2, which is an allowable pressure at which the examinee experiences extreme discomfort, the gas in the stomach 100 is not emitted via belching. The second pressure P2 is determined as appropriate according to the examinee's conditions (age, gender, and the like), but is, for example, 25 mmHg or higher.

If the pressure reaches the second pressure P2 (S19: YES), the process proceeds to step S14.

As described above, the gas supply control method includes supplying gas from a gas supply source to a lumen of a subject at a first flow rate F1, measuring a pressure of the gas within the lumen, and when the pressure reaches a first pressure P1, supplying the gas to the lumen at a second flow rate F2 below the first flow rate F1.

The gas supply control system 1 includes a first tube 11 communicating with a lumen within a body of an examinee, a pressure sensor 14 disposed on the first tube 11 and configured to measure a pressure of gas, a second tube 21 communicating with the lumen and configured to supply the gas from a gas supply source to the lumen, a valve 25 configured to adjust a flow rate of the gas supplied to the lumen via the second tube 21, and a controller 15 configured to perform control of the valve 25 based on the pressure detected by the pressure sensor 14.

The gas supply control system 1 includes an endoscope 9 including a gas/liquid feeding pipe 92 and a forceps channel 91 that pass through an insertion portion 93, the insertion portion 93 being configured to be inserted into a lumen of a subject, a first tube 11 connected to communicate with the forceps channel 91, a pressure sensor 14 disposed on the first tube 11 and configured to measure a pressure of gas, a second tube 21 configured to supply the gas from a gas supply source to the lumen via the gas/liquid feeding pipe 92, a valve 25 configured to adjust a flow rate of the gas supplied to the lumen via the second tube 21, and a controller 15 configured to perform control of the valve 25 based on the pressure detected by the pressure sensor 14, wherein the controller 15 is configured to control the valve 25 to supply the gas to the lumen at a first flow rate F1, and when the pressure reaches the first pressure P1, supply the gas to the lumen at a second flow rate F2 below the first flow rate F1.

According to the present embodiment, a gas supply control method for performing an examination quickly and accurately, a gas supply control apparatus 10 for performing the examination quickly and accurately, and a gas supply control system 1 for performing the examination quickly and accurately can be provided.

The above described the gas supply control system 1 used for diagnosing GERD and esophageal achalasia, using the stomach 100 as an example of a lumen of the subject. However, the lumen is not limited to the stomach 100, but may be any lumen to which gas is fed, such as, for example, the large intestine.

### Modifications

Gas supply control systems 1A-1C described below are similar to the gas supply control system 1 and have substantially the same effects as the gas supply control system 1. For this reason, constituent elements having substantially the same functions as those of the gas supply control system 1 will be denoted by the same reference numerals, and descriptions thereof will be omitted.

### Modification 1

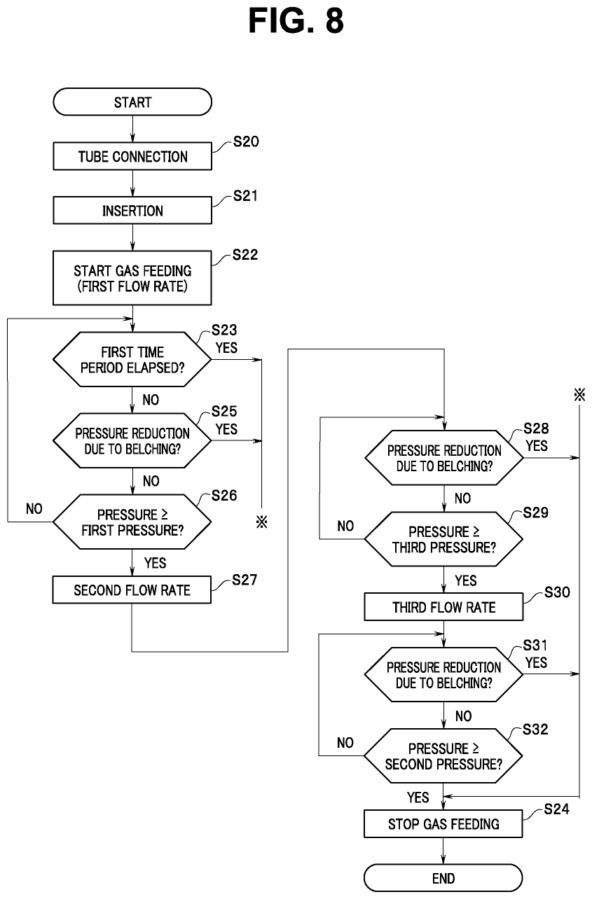

A gas supply control method for the gas supply control system 1A of the present modification example will be described along the flowchart of FIG. 8.

### Steps S20 to S28

Since steps S20 to S28 are substantially the same as steps S10 to S18 described in FIG. 3, the descriptions thereof will be omitted.

### <Step S29> Pressure≥Third Pressure?

Gas feeding at the second flow rate F2 continues until the pressure reaches a third pressure P3 (S29: NO). The third pressure P3 is greater than the first pressure P1 and less than the second pressure P2. The third pressure P3 is set as appropriate, and is, for example, 20 mmHg.

### <Step S30> Third Flow Rate

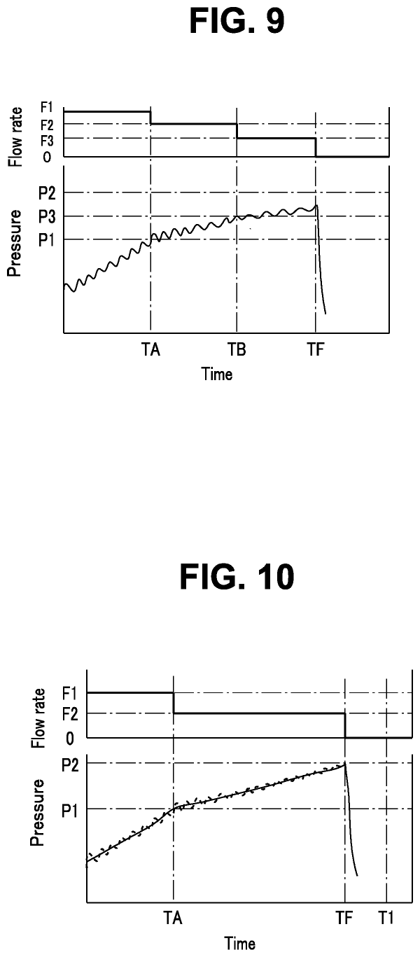

When the pressure reaches the third pressure P3 (S29: YES), gas is supplied at a third flow rate F3 below the second flow rate F2, as shown in FIG. 9. The third flow rate F3 is, for example, 0.1 ml/s to 1.0 ml/s.

For accurate diagnosis, for example, the third flow rate F3 may preferably be less than 75% of the second flow rate F2, and may particularly preferably be less than 50%.

### Step S31 to S32

Since steps S31 to S32 are substantially the same as steps S18 to S19 described in FIG. 3, the descriptions thereof will be omitted.

The gas supply control system 1A can perform a diagnosis even more accurately than the gas supply control system 1.

As already described, pressure thresholds (the first pressure P1, the second pressure P2, the third pressure P3) are set as appropriate. The pressure thresholds may be set by the user each time an examination is performed, or a plurality of sets of pressure thresholds may be stored in advance in the memory 16 for user selection.

### Modification 2

As already described, gastric internal pressure reflects respiratory variation, and rises during inspiration relative to expiration. For this reason, the measured pressure includes biological oscillations that repeat fluctuations (increases and decreases) according to the respiratory cycle.

In a gas supply control system 1B of the present modification example, as shown by the solid line in FIG. 10, the flow rate is controlled using an average pressure obtained by canceling the fluctuations (increases and decreases) of the pressure (dashed line) that fluctuates (increases and decreases) at substantially constant cycles.

For example, by using a low-pass filter, the gas supply control system 1B cancels the fluctuations (increases and decreases) of pressure in short-period cycles.

The controller 15 may cancel the fluctuations (increases and decreases) by performing a calculation process using moving average, least squares method, or the like. For example, the controller 15 may calculate an approximation formula using pressure data acquired over a predetermined time range, predict pressure changes based on the approximation formula, and perform control. If the approximation formula is a linear equation (pressure P=a*time T+b), the constants a and b are calculated. The constant a corresponds to the slope of the straight line (rising rate).

In control based on the approximation formula, the controller 15 performs predetermined control before the pressure detected by the pressure sensor 14 reaches a threshold pressure, or does not perform the predetermined control even if the pressure detected by the pressure sensor 14 reaches the threshold pressure.

For pressure data used for calculating the approximation formula, in order to reduce errors, it may be preferable to use data in a range of at least two cycles of increase and decrease.

The fluctuations (increases and decreases) of pressure may be cancelled by acquiring, prior to starting diagnosis, data on patterns of pressure changes (amounts of increases and decreases, and cycles) due to the examinee's breathing, and subtracting this change pattern data from the acquired pressure data.

By canceling the effects of biological oscillations, the gas supply control system 1B enables more accurate diagnosis.

### Modification 3

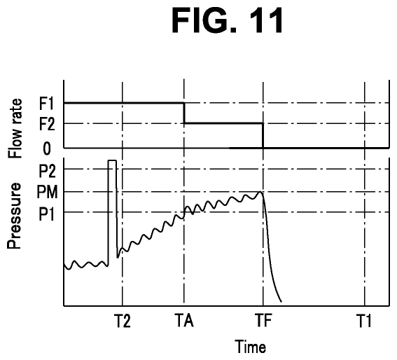

As shown in FIG. 11, in the gas supply control system, the rising rate of internal pressure is small immediately after gas feeding starts, and the internal pressure may thereafter temporarily increase significantly. The cause of this phenomenon is unclear.

In a gas supply control system 1C of the present modification example, the supply of gas at the first flow rate F1 continues until a time period during which gas is supplied at the first flow rate F1 reaches a second time period T2. It should be noted that the controller 15 may start control based on pressure and display pressure changes on the display 31 after the rising rate of the internal pressure becomes less than a predetermined rising rate and has stabilized.

The gas supply control system 1C may inhibit malfunction caused by temporary increases in internal pressure.

The present disclosure is not limited to the above-mentioned embodiments, etc., and various modifications, combinations, and applications are possible without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), microcontrollers, digital signal processors (DSPs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, ROM, or other storage media) that stores data, software, firmware, or instructions contributing to the processing operations. Accordingly, the processing circuitry may be implemented as a specialized hardware circuit having fixed logic, a programmable circuit executing instructions stored in an internal or external memory, or any combination thereof. The processing circuitry may be configured as, or include, one or more processors. Thus, the term “processor” used in the description of embodiments is to be understood as a specific example of the processing circuitry or as a component included within the processing circuitry. Furthermore, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

In this specification and the appended claims, the singular forms “a,” “an,” and “the” include plural referents unless the context clearly dictates otherwise. For example, reference to “a recess” includes a single recess as well as a plurality of recesses.

Terms of degree, such as “substantially,” “about,” “approximately,” and “generally,” as used herein, represent a reasonable amount of deviation from the specified value or state such that the end result is not significantly changed. These terms should be construed as including a deviation of at least ±5% or ±10% of the modified value, or a deviation recognized by a person of ordinary skill in the art, unless otherwise indicated.

As used herein, geometric, spatial, or directional terms such as “parallel,” “perpendicular,” “orthogonal,” “intersecting,” “polygon,” or specific angles (e.g., “45 degrees”) are not intended to require strict mathematical or geometric exactness. Rather, these terms should be understood to encompass substantial approximations, manufacturing tolerances, and practical variations recognized by those skilled in the art, unless otherwise explicitly claimed.

In the present disclosure, terms such as “same shape,” “uniform shape,” or “common shape” refer to having the same general profile, contour, or topological structure (e.g., both being T-shaped), and do not necessarily require identical dimensions, sizes, scales, or proportions, unless explicitly stated otherwise.

Non-limiting examples according to aspects of the present disclosure will be described in the following clauses:
Clause 1: A gas supply control method comprising:
   supplying gas from a gas supply source to a lumen of a subject at a first flow rate;
   measuring a pressure of the gas within the lumen;
   supplying the gas to the lumen at a second flow rate below the first flow rate when the pressure reaches a first pressure; and
   stopping supply of the gas to the lumen when the pressure reaches a second pressure greater than the first pressure.
Clause 2: The gas supply control method according to clause 1, further comprising:
   stopping supply of the gas to the lumen when a time period during which the gas is fed at the first flow rate reaches a predetermined time period.
Clause 3: The gas supply control method according to clause 1, further comprising:
   supplying the gas to the lumen at a third flow rate below the second flow rate when the pressure reaches a third pressure greater than the first pressure and less than the second pressure.
Clause 4: The gas supply control method according to clause 1,
   wherein the second flow rate is less than 75% of the first flow rate.
Clause 5: The gas supply control method according to clause 1,
   wherein the first pressure is a gastroesophageal reflux disease (GERD) diagnostic pressure, and
   wherein the second pressure is an allowable pressure.
Clause 6: The gas supply control method according to clause 3,
   wherein the third flow rate is less than 75% of the second flow rate.
Clause 7: The gas supply control method according to clause 1, further comprising:
   calculating a rising rate of the pressure, and
   supplying the gas to the lumen at the second flow rate when the rising rate reaches a predetermined rising rate, even if the pressure is equal to or less than the first pressure.
Clause 8: The gas supply control method according to clause 1, further comprising:
   controlling using an average pressure obtained by canceling fluctuations of the pressure that fluctuates at substantially constant cycles.
Clause 9: The gas supply control method according to clause 1, further comprising:
   continuing supply of the gas at the first flow rate until a time period during which the gas is supplied at the first flow rate reaches a particular time period.
Clause 10: A gas supply control method comprising:
   supplying gas from a gas supply source to a lumen of a subject at a first flow rate;
   measuring a pressure of the gas within the lumen;
   supplying the gas to the lumen at a second flow rate below the first flow rate when the pressure reaches a first pressure; and
   calculating a rising rate of the pressure and supplying the gas to the lumen at the second flow rate when the rising rate reaches a predetermined rising rate.
Clause 11: The gas supply control method according to clause 10, further comprising:
   stopping supply of the gas to the lumen when a time period during which the gas is fed at the first flow rate reaches a predetermined time period.
Clause 12: The gas supply control method according to clause 10, further comprising:
   stopping supply of the gas to the lumen when the pressure reaches a second pressure greater than the first pressure.
Clause 13: The gas supply control method according to clause 10, further comprising:
   supplying the gas to the lumen at a third flow rate below the second flow rate when the pressure reaches a third pressure greater than the first pressure and less than the second pressure.
Clause 14: The gas supply control method according to clause 10,
   wherein the second flow rate is less than 75% of the first flow rate.
Clause 15: The gas supply control method according to clause 10,
   wherein the first pressure is a gastroesophageal reflux disease (GERD) diagnostic pressure, and
   wherein the second pressure is an allowable pressure.
Clause 16: The gas supply control method according to clause 13,
   wherein the third flow rate is less than 75% of the second flow rate.
Clause 17: The gas supply control method according to clause 10, further comprising:
   controlling using an average pressure obtained by canceling fluctuations of the pressure that fluctuates at substantially constant cycles.
Clause 18: The gas supply control method according to clause 10, further comprising:
   continuing supply of the gas at the first flow rate until a time period during which the gas is supplied at the first flow rate reaches a particular time period.

What is claimed is:

1. A gas supply control method implemented by processing circuitry operatively coupled to a gas feeding assembly, the gas supply control method comprising:
causing the gas feeding assembly to supply gas to a lumen of a subject at a first flow rate;
obtaining a pressure of the gas within the lumen;
causing the gas feeding assembly to supply the gas to the lumen at a second flow rate less than the first flow rate when the obtained pressure is equal to or greater than a first pressure; and
causing the gas feeding assembly to cease supplying the gas to the lumen when the obtained pressure is equal to or greater than a second pressure greater than the first pressure.

2. The gas supply control method according to claim 1, further comprising:
causing the gas feeding assembly to cease supplying the gas to the lumen when a time period during which the gas is fed at the first flow rate reaches a predetermined time period.

3. The gas supply control method according to claim 1, further comprising:
causing the gas feeding assembly to supply the gas to the lumen at a third flow rate less than the second flow rate when the obtained pressure is equal to or greater than a third pressure greater than the first pressure and less than the second pressure.

4. The gas supply control method according to claim 1,
wherein the second flow rate is less than 75% of the first flow rate.

5. The gas supply control method according to claim 1,
wherein the first pressure is a gastroesophageal reflux disease (GERD) diagnostic pressure, and
wherein the second pressure is an allowable pressure.

6. The gas supply control method according to claim 3,
wherein the third flow rate is less than 75% of the second flow rate.

7. The gas supply control method according to claim 1, further comprising:
calculating a rising rate of the obtained pressure, and
causing the gas feeding assembly to supply the gas to the lumen at the second flow rate when the calculated rising rate exceeds a predetermined rising rate, even if the obtained pressure is equal to or less than the first pressure.

8. The gas supply control method according to claim 1, further comprising:
performing gas supply control using an average pressure obtained by canceling fluctuations of the obtained pressure occurring in substantially constant cycles.

9. The gas supply control method according to claim 1, further comprising:
causing the gas feeding assembly to continue supplying the gas at the first flow rate until a time period during which the gas is supplied at the first flow rate reaches a particular time period.

10. A gas supply control method implemented by processing circuitry operatively coupled to a gas feeding assembly, the gas supply control method comprising:
causing the gas feeding assembly to supply gas to a lumen of a subject at a first flow rate;
obtaining a pressure of the gas within the lumen; and
causing the gas feeding assembly to supply the gas to the lumen at a second flow rate less than the first flow rate when at least one of a first condition or a second condition is satisfied, wherein the first condition is a condition in which the obtained pressure is equal to or greater than a first pressure, and the second condition is a condition in which a rising rate of the obtained pressure exceeds a predetermined rising rate.

11. The gas supply control method according to claim 10, further comprising:
causing the gas feeding assembly to cease supplying the gas to the lumen when a time period during which the gas is supplied at the first flow rate reaches a predetermined time period.

12. The gas supply control method according to claim 10, further comprising:
causing the gas feeding assembly to cease supplying the gas to the lumen when the obtained pressure is equal to or greater than a second pressure greater than the first pressure.

13. The gas supply control method according to claim 12, further comprising:
causing the gas feeding assembly to supply the gas to the lumen at a third flow rate less than the second flow rate when the obtained pressure is equal to or greater than a third pressure greater than the first pressure and less than the second pressure.

14. The gas supply control method according to claim 10, wherein the second flow rate is less than 75% of the first flow rate.

15. The gas supply control method according to claim 10,
wherein the first pressure is a gastroesophageal reflux disease (GERD) diagnostic pressure, and
wherein the second pressure is an allowable pressure.

16. The gas supply control method according to claim 13,
wherein the third flow rate is less than 75% of the second flow rate.

17. The gas supply control method according to claim 10, further comprising:
performing gas supply control using an average pressure obtained by canceling fluctuations of the obtained pressure occurring in substantially constant cycles.

18. The gas supply control method according to claim 10, further comprising:
causing the gas feeding assembly to continue supplying the gas at the first flow rate until a time period during which the gas is supplied at the first flow rate reaches a particular time period.

19. A gas supply control system comprising:
a first tube configured to communicate with a lumen of a subject;
a pressure sensor disposed at the first tube and configured to measure a pressure of gas within the lumen;
a second tube configured to communicate with the lumen and configured to supply the gas from a gas supply source to the lumen;
a valve configured to adjust a flow rate of the gas supplied through the second tube; and
processing circuitry configured to:
control the valve to supply the gas to the lumen at a first flow rate;
obtain the pressure measured by the pressure sensor; and
control the valve to supply the gas to the lumen at a second flow rate less than the first flow rate when at least one of a first condition or a second condition is satisfied, wherein the first condition is a condition in which the obtained pressure is equal to or greater than a first pressure, and the second condition is a condition in which a rising rate of the obtained pressure exceeds a predetermined rising rate.

20. The gas supply control system according to claim 19,
wherein the processing circuitry is further configured to:
control the valve to cease supplying the gas to the lumen when at least one of a third condition or a fourth condition is satisfied, wherein the third condition is a condition in which the obtained pressure is equal to or greater than a second pressure greater than the first pressure, and the fourth condition is a condition in which a time period during which the gas is supplied at the first flow rate reaches a predetermined time period.
